(19) United States
(12) Patent Application Publication
(10) Pub. No.: US 2004/0214183 A1
(43) Pub. Date: Oct. 28, 2004

(54) Methods for global profiling gene regulatory element activity

(76) Inventors: Mary E. Warren Harper, Solana Beach, CA (US); Christopher C. Adams, San Diego, CA (US); Paul Labbart, San Diego, CA (US); Marc Ballivet, Geneva (CH)

Correspondence Address: WILMER CUTLER PICKERING HALE AND DORR LLP, 300 PARK AVENUE, NEW YORK, NY, 10022, US

(21) Appl. No.: 10/468,965

(22) PCT Filed: Feb. 28, 2002
(86) PCT No.: PCT/IB02/00589

Related U.S. Application Data
(60) Provisional application No. 60/272,753, filed on Mar. 1, 2001.

(51) Int. Cl. C12Q 1/68

(52) U.S. Cl. 435/6

(57) ABSTRACT

The present disclosure provides methods for profiling, in a global manner, the activity of gene regulatory elements in cells, preferably eukaryotic and prokaryotic cells. The methods include use of Fluorescence Polarization and other homogeneous assays, regulatory element activity profiling (REAP), and electrophoretic mobility shift assays. Cells may be in any state of metabolism, whether resting, growing, normal, mutant, diseased or differentiating.

### FIELD OF THE INVENTION

This invention relates generally to monitoring gene regulation. More specifically, this invention relates to methods for determining, in a comprehensive manner, gene regulatory element activity in cells. Even more specifically, the invention relates to global profiling gene regulation in eukaryotic and prokaryotic cells under various metabolic states of growth and/or differentiation and after exposure to external changes such as treatment with drugs.

BACKGROUND OF THE INVENTION

The following description includes information that may be useful in understanding the present invention. It is not an admission that any of the information provided herein is prior art, or relevant, to the presently claimed inventions, or that any publication specifically or implicitly referenced is prior art.

In recent years, gene expression and specifically the regulation of gene expression have generated intense interest. This is because gene regulation occupies the fundamental controlling center of cellular growth, differentiation, and development. Similarly, aberrant gene regulation has been recognized for playing a leading role in the onset and/or progression of many disease states. In light of this recognized relationship between gene regulation and cellular status, numerous technologies have been tested for their respective capabilities for identifying genetic differences between cell types or differences that result from various cell treatments. Such technologies have generally sought to uncover important specific genetic targets associated with medically-relevant physiological changes in specific cell types.

For example, the standard serial analysis of gene expression (SAGE) methodology has been used to identify genes differentially expressed in human normal versus tumor endothelial tissue, while MRNA expression profiling, by standard cDNA hybridization to DNA microarrays followed by fluorescent detection of duplexes, has been successfully used to identify genes associated with T-cell activation as well as inflammatory disease-related genes. However, the difficulty in interpreting the data from these types of studies that results from experimental variability have been documented. Furthermore, these approaches do not identify groups of coordinately regulated genes within the population of differentially expressed genes nor do they elucidate any mechanisms or factors responsible for the differential expression;

With respect to understanding the mechanism of gene regulation, much less is presently understood. It is known, however, that gene regulation occurs at the level of transcription initiation. At this level gene sequences include promoter and enhancer sequences that bind transcriptional activator and repressor molecules that act, in part, to regulate the expression of the gene sequences associated therewith. Activator molecules have been observed to bind to DNA and recruit molecular transcription initiation machinery. With respect to some genes, such initiation machinery includes RNA Pol II and at least 50 other molecular components. Generally, the transcription initiation machinery includes proteins that bind DNA, cyclin-dependent kinases that regulate polymerase activity, and acetylases and other enzymes that modify chromatin structures. Thus, it can be understood that gene expression is controlled by many selective protein-protein and protein-nucleic acid interactions.

Presently, the understanding of the mechanisms surrounding gene expression regulation is limited in large measure due to a lack in the art of a complete set of molecular trancriptional regulators as well as a lack of understanding how these regulators interact and control components of the transcriptional machinery. Only a small fraction of such components involved in controlling the transcriptional machinery are known or even understood, and then only with respect to a small number of genes. Moreover, considering that cells must adjust their genetic expression to accommodate changes in environmental and metabolic conditions to provide for growth control, differentiation, and development, it is clear that very little regarding exactly how genomic expression is coordinated is even known.

Only recently has genome-wide expression monitoring become feasible. This feasibility has occurred in conjunction with the completion of the first draft sequence of the entire human genome and through the development of cDNA and high-density oligonucleotide microarray technologies. For example, such expression monitoring has typically used classical mRNA expression profiling to elucidate differences in gene expression in yeast cells when grown in various media. Such profiling has also revealed how yeast genomic expression is remodeled during metabolic shifts from fermentation to respiration. Similarly, expression profiling has been used to study human biological processes and human disease states of cancer cells, and to facilitate drug development.

However, the data generated using such MRNA expression profiling describes metabolic states of cells only at the level of each MRNA species among the tremendously large population of RNA species. Due to the complexity of the cellular status at any given metabolic state, data obtained by such mRNA-based expression profiling alone does not always produce significant new biological insights. Specifically, because of the present embryonic level of knowledge concerning genome-wide transcriptional regulation, it is difficult to understand how such genome-wide expression signatures transpire and what the importance is of such transitions in gene expression.

In one aspect of gene expression regulation, it has become understood that a primary factor in the control of coordinated gene expression is the level of activity of trans-acting protein molecules (transcription factors which act as activators and repressors) that bind DNA in a sequence-specific manner. By assessing such protein/DNA binding activity in a cell at any given time, one can gain useful insight into changes in gene expression that occur as a result of this protein binding activity. Presently, such activity has been assessed for specific individual protein/DNA interactions using protein/DNA crosslinking techniques, electrophoretic mobility shift assays (EMSA), and footprinting. However, these techniques all suffer from an inability to assay for binding activities of large populations of proteins simultaneously. Moreover, of the several methods, including SAGE, cDNA microarrays, and oligonucleotide-based chips, that have been employed to address examining the relationship between disease and gene expression patterns related to such diseases, there is still the inability to decipher which transcription factors are involved and how they change expression patterns due to the lack of available sensitive and efficient techniques for examining and quantifying intermolecular communication.

Given the still present and ongoing need in the art for methods for understanding in a global fashion the regulatory mechanism of gene expression in cells, especially those related to disease, we describe below an invention that permits global analysis (i.e. profiling) of any given cell wherein the transcriptional potential of such cell can be measured by examining the individual binding activities of an entire population of proteins (e.g., transcription factors) derived from a cellular extract. Embodiments of the invention will be understood by the foregoing descriptions and claims.

### SUMMARY OF THE INVENTION

The present invention provides methods for determining the global profile of gene regulatory element activity in a cell population. By “global profile” is meant the activity levels of gene regulatory elements in a cell population as determined by the extent of formation of specific CIS element/nucleic acid binding factor complexes. A global profile may comprise activity levels of those known CIS element/nucleic acid binding factor complexes or a portion thereof in the cell population studied. As additional CIS elements and nucleic acid binding factors are discovered, they may be added the gene regulatory element activity profiling analysis. By “gene” is meant a particular sequence of D NA in a genome (which may be discontinuous in the DNA) that encodes a particular protein or group of proteins.

By “regulatory element” is meant 1) a CIS element of defined nucleic acid sequence (or sequence motif) within a promoter or enhancer region that is capable of associating with an endogenous or exogenous nucleic acid binding molecule and is used by a cell in the transcription process, or 2) a nucleic acid binding factor comprising a transcription factor (also called a Trans factor) that can bind to a CIS element or family of CIS elements in a sequence-dependent manner, and is involved in the transcription process or is part of the transcriptional machinery of a cell.

By “regulatory element activity” is meant the binding of nucleic acid binding molecules (herein called Trans factors) to CIS elements in the process of regulating gene transcription. Such regulatory elements are determined to be active as a result of their ability to form specific DNA/protein complexes under appropriate binding conditions, whereby their activity is quantified by the extent of their binding together in specific sequence-dependent complexes.

By “active regulatory elements” are meant those CIS elements and Trans factors that form specific nucleic acid/protein complexes when a plurality of proteins from a cell or a portion of a cell is combined with a plurality of nucleic acid molecules under conditions where Trans factors can recognize their cognate CIS elements and bind to them. Complexes may comprise one CIS element plus one Trans factor, combinations of CIS elements and Trans factors, or combinations of CIS elements and Trans factors with other proteins such as co-activating proteins or other members of the transcription machinery.

As used herein, “regulate” or “modulate” refers to an ability to turn on or off or to alter the level of expression of a particular gene, i.e., up-regulate or activate, or down-regulate or repress expression. In the case of treatment with a drug compound or exposure to different external conditions, the gene may be up-regulated or down-regulated relative to the basal level of expression that would occur in the particular system (for example, a cell or an in vitro transcription system) without the particular treatment under the same conditions.

In a preferred embodiment, the global profiling methods of the invention comprise obtaining a protein extract containing Trans factors from the cell population to be profiled, and combining the protein extract with a plurality of nucleic acid molecules comprising at least two CIS elements under conditions that allow formation of specific CIS element/Trans factor complexes. Preferably, the plurality of nucleic acid molecules comprises more than two CIS regulatory elements, or a library of nucleic acids, each comprising at least two, and preferably different, CIS regulatory elements. The CIS element/Trans factor regulatory complexes are characterized according to specific type and extent of binding activity in order to determine which are active and to what level in the original cell population. Such characterization is accomplished by any number of methods, including sequencing of the nucleic acid molecules that bind one or more Trans factors, hybridization of the bound nucleic acid molecules to other known nucleic acid molecules for the purposes of identification, or other detection systems that directly allow visualization or identification of which CIS elements or larger regulatory regions in which they are located are bound by Trans factors. Preferably, protein extracts containing the Trans factors may comprise a nuclear extract, cellular extract, cytoplasmic extract, extract from cells used for expressing (producing) a particular biomolecule such as a protein, mitochondrial extract, or chroloplast extract. Proteins contained within the extracts may be full-length proteins, partial proteins or peptides.

Another aspect of the invention concerns comparing the global gene regulatory activity profiles for two different cell populations and determining which elements exhibit differential activity between the two populations. Such methods comprise comparing the type and/or quantity of active CIS element/Trans factor complexes so formed from one cell population with the active CIS element/Trans factor complexes formed from the other cell population. In a further embodiment, cell populations to be compared include different cell types within the same organism, the same cell type between different organisms, normal vs. diseased cells of the same type, normal vs. transformed cells of the same type, cells at different stages of differentiation or development, cells treated with an exogenous material such as a drug compound or biomolecule vs. untreated cells, cells exposed to two different compounds or biomolecules, cells exposed to a different external or internal condition vs. unexposed, cells exposed to two different external or internal conditions, or cells within a comparison comprised of more than two different cell populations, (each of these comparisons comprising a comparison of cell populations that are at two different physiologic and/or metabolic states). In a particularly preferred embodiment, profiles obtained for different metabolic or physiologic states are compared between cell populations (preferably cells of the same linage) in order to determine differences in gene regulatory activity and hence gene expression between the two populations.

In another embodiment, the method of the invention provides for placing nucleic acid molecules comprising known sequences, which may or may not include particular CIS elements, in locations in an array, such as in specific tubes, microtiter wells or on a microarray surface. In a preferred embodiment, the nucleic acid molecules are contacted with protein extracts comprising Trans factors, followed by testing to determine whether nucleic acid/protein binding has occurred. In another preferred embodiment, the specific CIS element/Trans factor complexes are detected by methods that determine when one of the components in the complex, i.e., the CIS element or the Trans factor, is in a bound state, such as by fluorescence polarization or another type of homogeneous assay. Other embodiments include direct sequencing of the bound DNA molecules and analysis for CIS elements within the nucleic acid molecules, biochemical characterization of the bound Trans factors, hybridization to the bound nucleic acid molecules using specific nucleic acid probes with either a separation step to remove unbound components or a homogeneous assay format, other separation methods based upon molecular size such as capillary electrophoresis, and detection using antibodies directed against proteins associated with regulating transcription or certain chromatin structures.

In preferred embodiments, the methods of the invention employ libraries of nucleic acid molecules. In another embodiment, the library may comprise a population of nucleic acid molecules containing known CIS elements that bind Trans factors. Alternatively, the library may comprise nucleic acid molecules that may or may not contain CIS elements that bind Trans factors. Certain preferred embodiments include nucleic acid molecules that are found in genomic DNA or representative of genomic DNA from any animal, plant, bacteria, archaebacteria or virus. Alternatively, the nucleic acid sequences may be random. Nucleic acid molecules may also contain modified nucleotides, for example, methylated nucleotides, as well as, or alternatively, nucleotide analogs and derivatives. Nucleic acid molecules may be synthetic or isolated from cells, varying in length from about 4 to about 1000 nucleotides in length, comprise purified DNA, partially-purified DNA or unpurified DNA, and may comprise DNA within chromatin, a chromosome, or chromosome segment.

Cells from which protein extracts may be obtained include animal cells, plant cells, fungal cells, Archaea cells, and bacterial cells. Preferred animal cells include avian, bovine, canine, equine, feline, fish, human, murine, ovine, porcine, and primate cells. Other preferred cells and cell-like structures include pathogens such as viruses, bacteria, parasites and other microorganisms. Such cells may be obtained from in vivo or in vitro (including ex vivo) sources. Such cells may be normal, diseased, transformed, infected with a virus, pathogen or other exogenous organism, transfected or transformed with an exogenous gene, portion of a genome or genome, treated so as to represent a particular state of typical or a typical growth or maintenance, or represent a particular stage of development.

In a further embodiment, the DNA molecules and/or the proteins may be labeled with tags for detection (such as comprising fluorescence, radioactivity, chemiluminescence, bioluminescence, antigens detectable by antibodies, antibodies detectable by antigens, and other identifier molecules such as beads that can be specifically identified).

Preferably, the methods of the invention are performed in vitro, preferably in a high throughput format, meaning that more than about 10, preferably, more than about 100, 1,000, or 10,000 elements are profiled at once. The format may include an array, where either specific DNA oligonucleotides or combinations thereof are located in specific locations, such as microtiter plates, slides, gels, columns, microarrays, tubes or chips. Within each plurality of regulatory element components, individual DNA oligonucleotides or proteins may be located in separate and distinct locations. The format may also include arrays or other solid supports containing detection elements for CIS/Trans complexes, such as antibodies that bind to proteins associated with transcription or chromatin structures, or nucleic acid molecules that bind to CIS sites.

In still other embodiments, the global gene regulatory element activity profiling may be used in a variety of applications. 1) The status of gene expression within each particular cell population can be determined by analyzing which CIS site/Trans factor complexes are detected in the cell populations studied. Complexes that can be detected are most likely to be regulating specific gene expression, and the groups of genes regulated by each complex can be determined. This information can be used to define the groups of coordinately expressed genes that have changed in their expression patterns between two cell populations of interest. 2) The effects of exogenous materials on cells related to activities such as efficacy, mechanism of action, toxicity and resistance can be determined by comparing the profiling results between treated and untreated cell populations or between cells treated with a particular exogenous material versus a reference or otherwise known material. 3) The effects of altering the external or internal environment of cells, such as growth conditions, maintenance conditions, and toxic conditions can be determined by comparing the profiling results obtained from cell populations exposed or not exposed to various conditions, among cell populations exposed to a variety of conditions, or between cells exposed to a particular condition versus a reference or otherwise known condition. 4) The effects of conditions that place cells under different states, such as stationary vs. growth phases or growth at different rates can be determined by comparing profiling results from cells placed under different states or from cells under a reference or otherwise known state. 5) Approaches can be developed to alter gene regulation, using molecules such as CIS site, Trans factor or co-activator inhibitors, inducers or analogs, from knowing which CIS/Trans complexes are active within the cell population of interest. 6) The sets of coordinately regulated genes that are controlled by the gene regulatory elements found to be active by profiling can be determined using methods including but not limited to knocking in (supplementation, for example, by over-expressing the cDNA for the transcription factor) or knocking out (for example, by CIS site decoys, antisense to the transcription factor RNA, or RNAi) the particular CIS site/Trans factor activities, or direct sequence analysis of the CIS sites relative to the genes. 7) The genes regulated by specific regulatory elements can then be studied for their RNA expression patterns via any method typically used for RNA profiling. 8) The genetic regulatory circuitry, comprising the differentially expressed genes and their regulatory elements, can be defined using information gained from global gene regulatory element activity profiling. 9) Novel, previously unknown gene regulatory elements can be discovered using appropriate analysis systems of the profiling data, including but not limited to clustering analysis of the nucleic molecules that bind one or more Trans factors, and detection of which Trans factors bind to novel CIS sites. 10) Genes encoding novel Trans factors can be studied by RNA expression analysis to determine cell populations in which these Trans factors are present. 11) Active gene regulatory elements important in the particular cell population or disease of interest, such as promoters, enhancers, CIS sites and Trans factors can be determined by analyzing the global gene regulatory element activity profiling results for the cell population of interest. 12) Genes whose gene products can be targeted for development of therapeutic drugs or biomolecules, or diagnostic or pharmacogenomic markers, can be identified by analyzing the global profiling results in combination with other information including but not limited to the coordinately regulated gene sets and genetic regulatory circuitry. Similarly, diagnostic or pharmacogenomic tests can be developed.

With respect to exogenous materials, such materials include test compounds selected from the group consisting of a small organic molecule, a lipid, a carbohydrate, a peptide, a polypeptide, a mutant polypeptide, and a nucleic acid. Alternatively, such exogenous material may also comprise a plurality of test compounds. Alternatively, a variety of parameters may be screened, for example, different compound concentrations, nuclear extracts generated after different times following compound addition, etc. Preferably, the regulatable gene is a marker gene, such as a gene encoding a luciferase or green fluorescent protein.

With respect to coordinately expressed genes, sets of coordinately regulated genes determined by global regulatory element activity profiling can be studied further for expression levels. In a preferred embodiment, sets of genes regulated by the same regulatory elements can be profiled for RNA expression differences using methods such as RNA expression profiling.

Yet another aspect of the invention concerns kits for determining the global gene regulatory element profiles of cells. Another embodiment includes diagnostic or pharmacogenomic test kits. Also included are arrays of various types, such as microtiter or other micro arrays of DNA molecules, such as CIS elements, or proteins, such as Trans factors, for determining global gene regulatory element activity profiles. BRIEF DESCRIPTION OF THE DRAWINGS

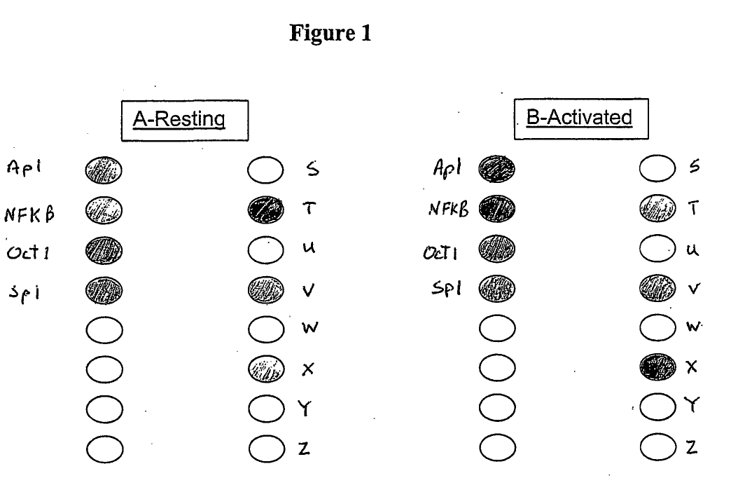

FIG. 1 shows a scheme for profiling regulatory element activity using fluorescence polarization.

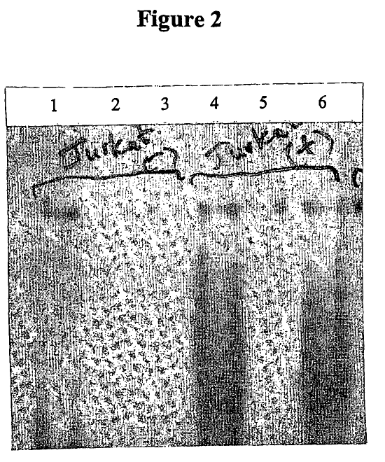

FIG. 2 is an electrophoretic mobility shift assay (EMSA) wherein nuclear extracts obtained from resting or TPA-activated Jurkat cells were used in separate binding reactions containing a ³²P-labeled oligonucleotide comprising a binding site for NF-kB. As shown in lanes 4 and 6, there is a significant increase in the gel-shifted material (DNA/protein complexes) from the activated Jurkat cells when no competitor (lane 4) or mismatched competitor (lane 6) was included. In contrast, matched competitor oligonucleotide to the NF-kB site prevented observation of specific NF-kB complexes (lane 5). Also, no increase in gel-shifted material was observed when the nuclear extract was from resting Jurkat cells, regardless of whether the reaction also contained no competitor (lane 1), matched competitor (lane 2) or mismatched competitor (lane 3). These results demonstrate that NF-kB CIS/Trans complexes are differentially present in Jurkat cells that have been activated.

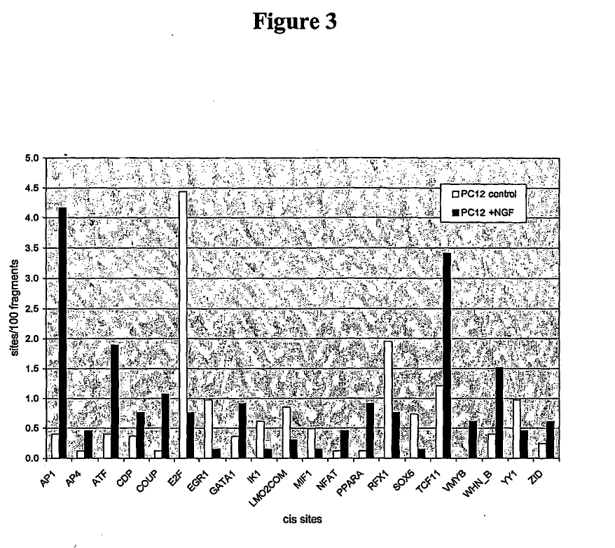

FIG. 3 is a graph wherein bars indicate the percentage of DNA fragments containing selected CIS sites that were isolated in binding reactions containing nuclear extracts from either untreated (white bars) or NGFbeta-treated (black bars) PC12 cells. The graph represents partial regulatory element activity profiles for both cell populations since other CIS element/nucleic acid binding factor complexes were also observed but not included in the graph. As indicated, the profiles of the two cell states are markedly different from one another.

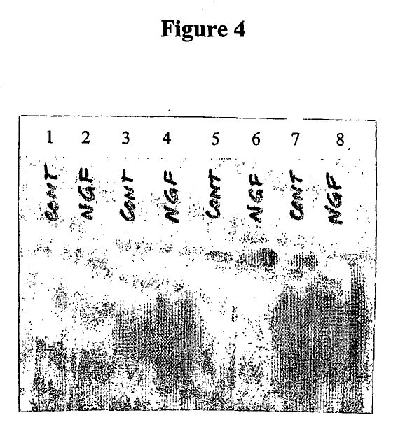

FIG. 4 is an electrophoretic mobility shift assay (EMSA) wherein nuclear extracts obtained from PC12 cells were either untreated or treated with NGFbeta in separate binding reactions containing a ³²P-labeled oligonucleotide comprising a binding site for a specific transcription factor. As shown in (lanes 3 and 4), there is a significant increase in the gel-shifted material (DNA/protein complexes) of the NGF-treated cells when the oligonucleotide was specific for the AP-1 binding site. In contrast, no increase in gel-shifted material was observed when the oligonucleotide was specific for the OCT1 binding site (lanes 7 and 8). Such results demonstrate that AP-1 is differentially activated in the NGF-treated cells, and that OCT1 CIS/Trans complexes are present in both cell populations but there is no activity differential between the NGF-treated and untreated cells. Lanes 1-2 and 5-6 lanes are from binding reactions that did not include intact nuclear extracts.

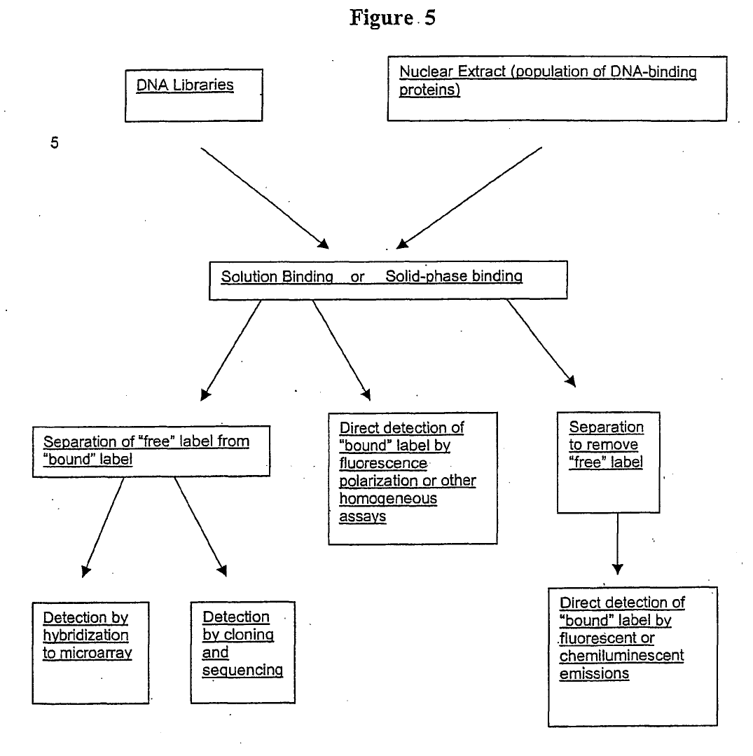

FIG. 5 shows a flow chart for regulatory element activity profiling methodology.

### DETAILED DESCRIPTION OF THE PREFERRED EMBODIMENTS

The present invention provides novel methods for performing global profiling of gene regulatory element activity in any cell population and determining differences in gene regulatory element activity between two or more cell populations.

Gene regulatory elements are comprised of cis acting nucleic acid elements (CIS elements) and the nucleic acid binding factors (transcription factors or Trans factors) that selectively bind such elements to regulate gene expression involved in all aspects of cell and organismal growth and development. Thus, CIS elements and Trans factors, when specifically bound together in sequence-dependent complexes, comprise an important aspect of the gene regulatory mechanisms directing cell modulation and tissue growth, development, pathogenesis, regeneration and repair by altering, enhancing, and/or reducing the expression of the genes they regulate.

The term “cis acting nucleic acid element” (CIS element) refers to a single-stranded or double-stranded RNA or DNA sequence that can be selectively bound by nucleic acid binding factors to regulate one or more genetic activities of a nucleic acid sequence present on the same molecule. As used herein, a CIS element is a DNA sequence that is associated directly with any specific gene and can be bound by a DNA-binding protein that is 1) used by a cell in transcription, 2) is part of the transcriptional machinery, or 3) is an exogenous or synthetic molecule that can serve the function of an endogenous DNA-binding molecule. Preferred CIS elements include nucleic acid sequences that occur endogenously in association with the gene whose transcription is to be regulated. CIS elements may be those previously described, e.g., in the scientific literature, in databases or known to other investigators, or those that are novel and detected and analyzed as a result of the global profiling method of the instant invention. CIS elements may comprise nucleic acid sequences within promoters and enhancers. By “promoter” is meant the minimum sequence necessary to initiate transcription of a gene by an RNA polymerase, for example, in eukaryotic cells, RNA polymerase I (which transcribes ribosomal RNA (rRNA) in eukaryotic cells), RNA polymerase II (which transcribes messenger RNA (mRNA) in eukaryotic cells), and RNA polymerase m (which transcribes transfer RNA (tRNA) in eukaryotic cells). By “enhancer” is meant a CIS-acting sequence that increases the utilization of a eukaryotic proinoter.

CIS elements involved in regulating gene expression are found in a variety of different types of DNA as well as at diverse genetic loci. Certain of these CIS elements, for example, TATA boxes, are found in a majority of genes. Other CIS elements, for example, hormone response elements, are localized within the nucleic acid sequence it regulates, or upstream or downstream thereof.

CIS elements contemplated for use in the methods of the invention can also comprise a diverse population of nucleic acid molecules. As used herein, the term “diverse population of nucleic acid molecules” refers to a composition comprising a plurality of different isolated polynucleotide nucleic acid molecules that potentially contain CIS elements. The diverse population of nucleic acids used in the methods of the invention can be of a variety of different types, structures and topology. The choice of nucleic acid type, structure and topology will depend on the needs of the methods used to perform the global profile as well as the desired results retrieved from such profile. For example, the diverse populations of nucleic acids of the invention can include double-stranded or single-stranded DNA, as well as linear, circular or branched nucleic acid molecules. Nucleic acid molecules of interest may be inserted in standard cloning vectors such as plasmids or viral genomes.

The methods of the invention contemplate using nucleic acid binding factors. By “nucleic acid binding factor” is meant a factor that selectively binds a cis acting nucleic acid element to modulate a genetic activity of a nucleic acid or group of nucleic acids. Preferably, a nucleic acid binding factor is a transcription factor (also called a Trans factor) and comprises a DNA-binding protein that 1) binds to a CIS element, and 2) is used by a cell in transcription. A Trans factor can interact covalently or non-covalently with other factors to form a complex that binds a CIS element. The factors within such a binding complex are also included within the term “Trans factor”. Some Trans factors within a complex of Trans factors can contact a CIS element directly. Other Trans factors within a complex of Trans factors do not contact a CIS element directly, but can contact one or more other Trans factors.

A Trans factor can be a polypeptide or a polypeptide that is modified, for example, by phosphorylation or addition of one or more carbohydrates, nucleotides, nucleic acids, cofactors or lipids. A Trans factor can also be a non-proteinaceous molecule, such as a lipid, carbohydrate or nucleic acid, or any combination thereof. Use of such Trans factors in connection with the methods of the invention may comprise a diverse population of nucleic acid binding factors. As used herein, the term “diverse population of nucleic acid binding factors” means a composition containing a plurality of different Trans factors. The greater the number of different factors within the population, the greater the diversity of the population. A population of Trans factors can be of low diversity for certain applications of the method. For example, a population of nucleic acid binding factors of low diversity can include, for example, 2, 3, 4, 5, 6, 7, 8, 9, between about 10 and 20, between about 21 and 50, or between 51 and 100 different nucleic acid binding factors. A population of nucleic acid binding factors of higher diversity can include more than about 100, more than about 10³, or more than about 10⁴ different nucleic acid binding factors. Such diversity may, for example, originate in all Trans factors found in a cellular extract. As with the diverse populations of isolated nucleic acid molecules, the members within a diverse population of nucleic acid binding factors can be known, unknown or partially known so long as some of the factors are different.

The methods of the invention are applicable to the profiling of gene regulatory element activity of a wide variety of nucleic acid types and sizes, and from any organism.

In one embodiment, a library or plurality of nucleic acid molecules, each comprising at least one and preferably different CIS elements, is combined with a protein extract from the cell population to be profiled under conditions that allow formation of specific DNA/protein complexes (CIS element/Trans factor complexes) under appropriate conditions. Gene regulatory elements are determined to be active as a result of their ability to form such CIS element/Trans factor complexes. The specific DNA/protein complexes are each quantified for binding activity as a measure of gene regulatory element activity in the original cell population. Complexes formed may comprise one CIS element plus one Trans factor, one CIS element plus more-than one Trans factor, more than one CIS element plus one Trans factor, or more than one CIS element plus more than one Trans factor. Complexes may also comprise a combination of one or more CIS elements plus one or more Trans factors plus one or more co-activating molecules. Complexes may further comprise a combination of one or more CIS sites plus one or more Trans factors plus one or more members of the transcription machinery.

Another aspect of the invention concerns comparing the global gene regulatory activity profiles for two different cell populations and determining which elements exhibit differential activity between the two populations. Such methods comprise comparing the quantity of active CIS element/Trans factor complexes so formed from one cell population with the active CIS element/Trans factor complexes formed from the other cell population. In a further embodiment, cell populations to be compared include different cell types within the same organism, the same cell type between different organisms, normal vs. diseased cells of the same type, normal vs. transformed cells of the same type, cells at different stages of differentiation or development, cells treated with an exogenous material such as a drug compound or biomolecule versus untreated cells, cells exposed to two different compounds or biomolecules, cells exposed to a different external or internal condition versus unexposed, cells exposed to two different external or internal conditions, or cells within a comparison comprised of more than two different cell populations. In a particularly preferred embodiment, profiles obtained for different metabolic or physiologic states are compared between cell populations (preferably cells of the same linage) in order to determine differences in gene regulatory activity and hence gene expression between the two populations.

In other embodiments, cells to be tested for gene regulatory element activity may be in any state of metabolism or under any physiologic condition. For example, in one aspect, cells are treated (in vitro or in vivo) with one or more compounds that affect the cell's metabolism or physiologic status. Such compounds may be administered at one or more concentrations. The cells may also be pre-treated with other molecules prior to adding the particular compound of interest. Alternatively, other compounds may be added after the cells are exposed to the compound(s), and/or environmental conditions under which the cells are grown may be changed. Following the addition of such compounds and/or alteration in environmental conditions, the cells of interest are globally tested for changes in their gene regulatory element activity.

In preferred embodiments, the methods of the invention employ libraries of nucleic acid molecules. In another embodiment, the library may comprise a population of nucleic acid molecules containing known CIS elements that bind Trans factors. Alternatively, the library may comprise nucleic acid molecules that may or may not contain CIS elements that bind Trans factors. In still other preferred embodiments, the nucleic acid molecules used in the methods according to the invention will each contain at least one CIS element. In certain embodiments, the oligonucleotides comprise 2, 3, 4, 5, 6, 7, 8, 9, or 10 CIS elements. Each nucleic acid molecule may contain a different CIS element or some CIS elements may be in common among multiple nucleic acid molecules. Such nucleic acid molecules may comprise defined nucleic acid sequences. Certain preferred nucleic acid molecules comprise nucleic acid sequences that are representative of a genome. Other preferred nucleic acid molecules comprise nucleotide sequences found in genomic DNA.

Alternatively, the nucleic acid sequence may be random. A “defined nucleic acid sequence” refers to a specific sequence of nucleotides, and is typically represented in the 5′ to 3′ direction using standard single letter notation, where “A” represents adenine, “G” represents guanine, “IT” represents thymine, and “C” represents cytosine. It will be appreciated that a nucleic acid molecule having a defined nucleotide sequence may include a different nucleotide at the same position, i.e., is degenerate at that position, with respect to one or more positions in the particular sequence. Degenerate bases may be represented by any suitable nomenclature, for example, that which is described in World Intellectual Property Organization Standard ST.25 (1998), Appendix 2. Random nucleic acid molecules may also comprise nucleotide sequences representative of a genome. For example, a nucleic acid molecule may comprise the same bias for nucleotide representation as a particular genome.

Nucleic acid molecules may be synthetic or isolated from cells, varying in length from about 4 to about 1000 nucleotides in length, comprise purified DNA, partially-purified DNA or unpurified DNA, and may comprise DNA within chromatin, a chromosome, or chromosome segment. Oligonucleotides may be representative of or a part of a genome comprising human, mammalian, vertebrate, animal, plant, fungi, eukaryotic, prokaryotic or viral genomes. Nucleic acid molecules may contain modified nucleotides, for example, methylated nucleotides, as well as, or alternatively, nucleotide analogs and derivatives. Nucleic acid molecules may also comprise a first amplification primer site upstream of the CIS element and a second amplification primer site downstream of the CIS element.

Cells from which protein extracts may be obtained include animal cells, plant cells, fungal cells, Archaea cells, and bacterial cells. Preferred animal cells include avian, bovine, canine, equine, feline, fish, human, murine, ovine, porcine, and primate cells. Other preferred cells and cell-like structures include cells infected with pathogens such as viruses, bacteria, parasites and other microorganisms. Such cells may be obtained from in vivo or in vitro (including ex vivo) sources, including tissues, organs, or whole organisms. Such cells may be normal, diseased, transformed, infected with a virus, pathogen or other exogenous organism, transfected or transformed with an exogenous gene, portion of a genome or genome, treated so as to represent a particular state of typical or a typical growth or maintenance, or represent a particular stage of development. Cells may further include fibroblasts, epithelial, hematopoietic, CNS-derived, bone-derived, myocytes, stem cells, basal cells, and the like.

In certain embodiments, the methods of the invention employ assay formats that use diverse populations of nucleic acid molecules comprising one or more CIS elements and diverse populations of Trans factors. Such elements are used preferably in an array format such that different nucleic acid molecules containing different CIS elements or different Trans factors are positioned at separate locations on the array. In one embodiment, the method of the invention provides for placing nucleic acid molecules comprising known CIS elements in an array format, e.g., within specific tubes, within specific wells of microtiter plates, or on specific locations of a microarray. The nucleic acid molecules on the array are contacted with cellular extracts comprising Trans factors followed by testing whether nucleic acid/protein binding has occurred. In an alternative embodiment, a diverse population of nucleic acid molecules comprising a library of all possible protein binding sequences are placed on an array and tested for binding with Trans factors obtained from or within cellular extracts. Whether known CIS element-containing nucleic acid molecules or a library of all possible protein binding sequences are used, the nucleic acid molecules are contacted with protein extracts followed by testing for levels of protein binding to the nucleic acids. In preferred embodiments, such testing is carried out by determining changes in the polarization of a fluorescent reference tag using fluorescence polarization over a predetermined time period. This technique provides for direct, nearly instantaneous measurement of a labeled molecule's (tracer's) bound/free ratio, even in the presence of free tracer. Fluorescence polarization is a measure of the time-averaged rotational motion of fluorescent molecules. A fluorescent molecule, when excited by polarized light, will emit fluorescence with its polarization primarily determined by the rotational motion of the molecule. Since molecular rotation is inversely proportional to the molecular volume, the polarization is in turn related to the molecular size. A small molecule rotates fast in solution and exhibits a low value of polarization whereas a large molecule exhibits a higher polarization because of its slower motion under the same conditions. Thus, changes in fluorescence polarization can reflect the association or dissociation between molecules of interest including transcription factors and small DNA fragments bound to their cognate binding sites.

In another embodiment, the specific CIS element/Trans factor complexes may be detected by 1) direct sequencing of the bound DNA molecules and analyzing for CIS elements within the DNA sequences, 2) other methods that detect when one of the components in the complex, i.e., the CIS element or the Trans factor, is in a bound state, such as a homogeneous luminescent assay (e.g., amplified luminescent proximity homogeneous assay (ALPHA), 3) biochemical characterization of the bound Trans factors, 4) hybridization to the bound DNA molecules using specific nucleic acid probes such as a nucleic acid molecule containing a CIS element, e.g., acridinium ester-labeled probes in a homogeneous assay format, 5) other separation methods based upon molecular size such as capillary electrophoresis, and 6) detection using antibodies directed against proteins associated with transcription regulation or certain chromatin structures.

Preferably, the methods of the invention are performed in vitro, preferably in a high throughput format, meaning that more than about 10, preferably more than about 100, 1,000, or 10,000 elements are profiled at once. The format may include an array, where either specific DNA molecules or combinations thereof are located in specific locations, such as microtiter plates, slides, gels, columns, microarrays, tubes or chips. Within each plurality of regulatory element components, individual DNA molecules or proteins may be located in separate and distinct locations. The format may also include arrays or other solid supports containing detection elements for CIS element/Trans factor complexes, such as antibodies that bind to proteins associated with transcription or chromatin structures, or nucleic acid molecules that bind to CIS elements. Preferably, such methods are performed where the complexes are formed and/or detected in solution, on solid surfaces, on solid supports, in semi-solid media, in gels, in column matrices, in polymer formulations, in aqueous formulations, in organic solutions, or in nonorganic solutions.

In other embodiments, detection of gene regulatory element activity is by detection of changes in condition of labels either attached to the CIS element-containing DNA molecules or plasmids, or incorporated into proteins that bind such elements. For example, a radioactively labeled amino acid can be used. In other embodiments, the changes in fluorescence may be determined such as by fluorescent polarization. Other variations of this sort will be apparent to those skilled in the art upon reading this specification.

In preferred embodiments, extracts of cells of interest for testing are prepared and applied to the CIS element-containing nucleic acid molecules. In the methods of the invention, the nucleic acid molecules of the arrays are examined for binding of Trans factors to the CIS elements. In a particularly preferred embodiment, it is not necessary to remove cellular extract material containing unbound proteins prior to detecting the presence of proteins bound to the CIS elements. In embodiments where the CIS element-containing nucleic acids are in solution, although it is not necessary to remove unbound proteins of the extract, it is useful to separate bound complexes (i.e., CIS elements bound to Trans factors) from the unbound matter.

In some embodiments, nuclear extracts containing nuclear proteins, for example, activators, repressors, transcription factors, and proteins involved in chromatin structure formation, maintenance, and/or remodeling, are obtained from cells of interest (either before or after exposure to compounds or environmental conditions). Such extracts may be obtained at a single time point following exposure to the compounds, or at different times. Preferably, the nuclear extracts are combined with the CIS element-containing nucleic acids that may be presented either singularly, or in preferred embodiments, in the form of nucleic acid libraries. Depending on the particular assay, for example, an assay wherein the nucleic acids are in solution as opposed to on a fixed array, complexes formed by the binding of the CIS elements with nucleic acid binding proteins are separated from unreacted portions of the extract/library/mixture. For example, when the nucleic acids are in solution, complexes can be isolated as a group simultaneously for further processing and detection of individual CIS element/Trans factor complexes. In contrast, when the nucleic acids are bound to a solid support, e.g., as a nucleic acid library in the form of an array, labeled proteins that interact therewith can be detected directly. Those in the art will appreciate that an unlabeled Trans factor bound to its cognate CIS regulatory element can be detected in other ways, for example, using detectable antibodies or other epitope-specific moieties.

In further embodiments, results of assays are compared with one or more control assays. In certain preferred embodiments, the control assay concerns obtaining a protein extract from cells that have not been exposed to compounds or changes in environmental conditions, or that have been exposed to compounds under different conditions, for example, at different concentrations, or for differing periods of time, etc. In particularly preferred embodiments, the differences in the expression of Trans factors, as indicated by the differences in the makeup of CIS element/Trans factor complexes, provides data valuable for determining gene regulatory element activity. Moreover, such data is provided by the methods of the invention at a global level for any cell extract tested. Thus, not just specific and/or known regulatory elements are tested for activity, but all regulatory element activities are detected.

Notwithstanding the complexity of the results presented by such global profiling, the methods of the invention allow for deciphering data so retrieved. For example, in any one global profile, many regulatory elements are involved, such as those elements that regulate the expression of more than one gene, or numerous elements that regulate different genes. In preferred methods where nucleic acid molecules are presented in an array, particular regulatory elements are identified directly and the genes with which the regulatory elements are functionally associated will also be directly determined. By “functionally associated’ is meant those genes over which the CIS element has some regulatory influence, be it activation, repression, sequestering in chromatin, etc. In other examples, where libraries of regulatory elements are assayed having new CIS element sequences, databases listing Trans factors that bind thereto can be formulated to determine which genes the CIS element is proximal to in the genome. As is understood by the skilled artisan, such databases can include databases of genes whose expression is at least partially controlled by the CIS element. From such information, some or all of the genes whose expression may be influenced by a particular regulatory element can be identified. Accordingly, a nucleic acid array containing hybridization probes specific for some or all of the genes functionally associated with the particular regulatory element (or set of particular regulatory elements) can be prepared. Carried to its conclusion, a database of all regulatory elements and the genes whose expression they control can be developed.

Methods

1. Preparation of Nucleic Acids.

Pluralities of DNA molecules useful for global profiling and that comprise regulatory elements can be obtained in various ways. For example, they can be double-stranded oligonucleotides that comprise nucleotide sequences derived from genomic sequences. Such genome-representative oligonucleotides typically comprise about 25-200 base pairs, preferably 35-100 base pairs, even more preferably 45-50 base pairs of genomic DNA flanked by primer binding sites. In a preferred embodiment, at least one of the oligonucleotides of a duplex is biotinylated at either its 5′ or 3′ end, thereby allowing either the biotinylated oligomer or even the duplex to be extracted with strepavidin. In certain alternative embodiments, the region between the primer sequence binding sites can comprise a known CIS site or a synthetic nucleotide sequence, including a random sequence. For example, in certain embodiments, oligonucleotides containing 16-mer randomized sequences flanked by primer binding sites and labeled with biotin at one end can be employed. Chemical methods for attaching the detectable label biotin (i.e., biotinylating) are known in the art. See, e.g. Agrawal, Chapter 3 in Protocols for Oligonucleotide Conjugates, Volume 26, Humana Press, Totowa, N.J. 1994, pages 93-120 (see especially pages 108-109) and Chu et al, Chapter 5, Id., pages 145-165 (see especially page 157). Oligonucleotides and other nucleic acids can also be biotinylated using enzymatic systems such as, e.g., nick translation (E. coli DNA Polymerase I and Dnase I; Boyle, Section V of Chapter 3, in Short Protocols in Molecular Biology, Second Edition, Ausubel, et al. Editors John Wiley & Sons, New York, 1992 pages 341 to 3-44) or “tailing” reactions using terminal deoxynucleotidyl transferase (see, e.g., the LABEL-IT™ 3′ Biotin End Labeling Kit from CPG, Inc., Lincoln Park, N.J.).

The nucleic acids useful in global profiling may be those already known in the art, for example, all known CIS elements, e.g., those described in public databases including binding sites for known transcription factors such as Ap-1, CREB, NF-kB, etc. Such sequences may be synthesized as short oligonucleotides; they may also be labeled with a fluorescent moiety, and aliquoted into wells of microtiter plates such that each well contains a unique sequence, or arranged on a surface in the form of an array.

The nucleic acids may further comprise a random DNA library wherein oligonucleotides are designed having a fully randomized central segment flanked by two fixed but different sequences on either side. Typically, the randomized sequence is at least 10 nucleotides in length. The fixed sequences can contain, for example, a restriction site immediately next to the randomized region, such that the central region can be separated from the fixed regions for concatemerization, cloning and sequencing. In an alternative embodiment, the fixed sequences may further provide for primer annealing so that the sequence may be amplified, such as by PCR, for identification once it has been determined in an assay that such sequence was bound by a nucleic acid-binding protein.

Nucleic acid molecules useful for global profiling can also comprise genomic DNA libraries. Such libraries provide for access to CIS elements whether of known sequence or unknown sequence. Such genomic libraries can contain short regions of total genomic DNA, including all functional regions of the genome, and can be generated such as by random cleavage followed by cloning into vectors flanked by restriction enzyme and/or amplification primer sequences. They can also be synthesized using the genomic DNA as a template so that the short DNA molecules are representative of the genomic DNA. Further, such sequences may be amplified, tagged with a fluorescent moiety, and aliquoted into wells of a microtiter plate as described above for random sequence libraries.

Whether known, random, or genomic, the nucleic acid molecules useful for global profiling may be used in assays either in solution or on a solid surface. With respect to use of known CIS elements on a surface, individual nucleic acids containing specific CIS elements may be applied to a surface in an organized array so that specific CIS elements have a known position. With respect to nucleic acids containing randomly generated sequences and sequences generated from genomic sources, such sequences can be individually cloned followed by specific placement on an array or simply layered onto a surface. Where layered onto a surface, individual molecules may be globally assayed by any number of methods. For example, antibodies may be generated to either specific nucleic acid sequences, or may be generated to specific proteins. Following formation of nucleic acid/protein complexes, such antibodies may be employed to screen for such complexes. Specific methods for carrying out such screening are well understood by those of skill in the art.

In embodiments where CIS element containing nucleic acids are used in global profiling in solution, detection of complex formation with proteins of cellular extracts may be by use of, for example, an array of high affinity polyclonal, and preferably monoclonal antibodies, raised against either a portion of the nucleic acid containing the bound CIS element or the nucleic acid-binding proteins. In such embodiment, the protein is preferably a known protein. Further, such antibodies can be arrayed on a solid support in a manner analogous to the CIS element arrays. The results of binding of the antibodies to the CIS element/Trans factor complex may then be detected by any suitable technique, for example, by using several probes, e.g., one attaching to the nucleic acid and another attaching to the protein.

2. Cellular and Nuclear Extracts

Cellular and nuclear extracts can be prepared by any suitable method, including by hypotonic lysis on ice, pelleting of nuclei and extraction of proteins in high salt buffer, and then dialysis or dilution to 100 mM salt, and storage at −80° C.

3. Detection Methods

Various methods may be used in conjunction with the global profiling methods of the invention. For example, regulatory element activity profiling (REAP) represents an exemplary technique that can be used for elucidating specific CIS elements with significant binding activity. In this aspect, REAP involves contacting DNA molecules in a nucleic acid library with binding factors (Trans factors) in nuclear extracts of cells treated with a compound followed by separating CIS element/Trans factor complexes from other constituents of the nuclear extract/library reaction using electrophoretic mobility shift assays (EMSA). Meaningful data is derived by comparing the REAP results from cells treated versus not treated with the compound, or by comparing REAP results from cells treated with the compound for different periods of time, at different concentrations, or in the presence of other compounds. A brief description of REAP is provided below. Additional descriptions of alternative procedures useful in the practice of the invention are found in U.S. Pat. No. 6,100,035.

REAP reactions may be performed by combining in a test tube the following components to generate a binding reaction mixture containing CIS element-comprising DNA (1-2 ng), nuclear extract (5-10 ug), poly dI:dC (1 ug or 0.5 mg/ml), E. coli RNA (1 ug or 0.5 mg/ml), and buffer components (20 mM HEPES, 50-100 mM salt (KCl or NaCl), 0.2 mM EDTA, and 1 mM MgCl₂, 5-10% glycerol, 0.5 mM DTT). After combining the various components, the binding mixture is incubated (for example, from about 5 min to about 3 hr at room temperature).

An electrophoretic mobility shift assay (EMSA) represents a preferred embodiment for separating multiple sets of bound CIS element/Trans factor complexes from unbound nucleic acid molecules and/or proteins. Typically, separation, and gel shifting, is performed using a polyacrylamide gel (cross-linked to the degree desired, depending on the size of the DNA molecules used). Shifted complexes are excised from the gel and eluted. The eluted nucleic acids are then recovered, for example, using streptavidin-dynabead magnetic separation when biotinylated o-ligonucleotides are used, and further denatured in order to elute single strands by alkaline conditions. PCR (or another rapid nucleic acid amplification protocol) is then used to amplify the sequences. If desired, an additional round of complex formation, EMSA, nucleic acid isolation, and amplification may be performed. The further CIS element/Trans factor bound complexes can also be separated by EMSA. The bound DNA molecules can then be identified by sequencing, cloning, or hybridization techniques (for example, to a nucleic acid array comprising a plurality of CIS element-specific probes, wherein hybrids can be detected using a labeled second probe specific for a primer binding site).

Another aspect useful in connection with the present global profiling invention is the discovery of novel CIS elements for nucleic acid binding proteins. For example, random sequence DNA molecules or genomic DNA molecules, as described above, can be mixed with a population of cellular proteins in solution under conditions that promote sequence-specific protein/DNA interactions and the level of protein binding to each individual DNA molecule is measured by fluorescence polarization or a similar method for quantifying protein/DNA binding. Simultaneous binding of a known Trans factor to a known CIS element can be monitored by simultaneous fluorescent detection of two or even three distinguishable fluorescent tags. This control binding event can be used as an internal control for validating binding conditions and for quantifying the level of protein binding to the random DNA sequence molecule or genomic DNA molecule (unknowns) by comparison. Those random or genomic fragments that exhibit a significant level of protein binding, as determined by fluorescence polarization or other detection method, are then amplified and their sequence determined by standard molecular biology methods. These sequences are considered sites for sequence-specific protein binding. Comparison of their sequence with known CIS element sequences is then performed to determine if they belong to the class of known protein binding sites or are novel protein binding sites, i.e., they have no recognizable homology to known protein binding sites.

In other preferred embodiments for detection, global profiling the regulatory element binding activity present in any particular cellular extract may be carried out, for example, by the following described assay method. CIS element-containing molecules placed on an array at a density of one protein binding site per molecule and one CIS element sequence per location on the array is labeled with a fluorescent marker (FIG. 1). Such arrays are mixed with solutions containing populations of cellular proteins under conditions that promote sequence-specific protein/DNA interactions and the level of protein binding to each individual DNA molecule is measured by fluorescence polarization or a similar method for quantifying protein/DNA binding. The exact level of binding to each individual CIS element by proteins contained in different cellular protein populations is quantified and compared. This comparison then provides a profile of differing binding activities that are present in the cells used to prepare the protein populations. Simultaneous binding of proteins to CIS elements can be monitored by simultaneous fluorescent detection of two or more discernable fluorescent tags. For example, the CIS element specific for AP-1 protein binding could be added to two separate arrays. Nuclear protein extracts prepared from either resting (untreated) Jurkat cells or TPA/ionomycin-treated Jurkat cells could then be added to these two arrays such that proteins from resting cells are placed on the array with the AP-1 CIS binding site molecule and the proteins from TPA-treated Jurkat cells are on the other. The level of protein binding to the AP-1 site in each of the two extracts is then measured by fluorescence polarization. If binding occurs, the level of bound CIS element following addition of the extract will be significantly higher and therefore result in much higher measurements of fluorescent anisotropy than prior to extract addition. The precise level of AP-1 binding in both protein samples and thus the level of induction of AP-1 binding by TPA treatment can be measured. This approach, when carried out in parallel with hundreds or even thousands of different protein binding sites, can successfully profile the binding activities of all known and even unknown Trans factors within different cell types, which is indicative of global changes in patterns of gene expression.

As an example of such profiling, FIG. 1 shows labeled DNA molecules from the library placed in solution into individual wells of microtiter plates such that each well contains a unique sequence that is unknown (represented by letters S-Z) or one that is known to bind sequence-specific DNA-binding proteins (e.g., for example Ap-1, NF-kB, Oct-1 and Sp-1). The solution may also contain nonspecific “carrier” DNA and/or internal control DNA. Identical replicates (shown as plates A and B) of the microtiter plates are produced for each different protein population (e.g. cellular extract) to be compared. For example shown here is comparison of resting (A) and TPA-activated (B) Jurkat cells.

Nuclear extracts containing populations of DNA-binding proteins are added to arrays of DNA molecules possessing binding sites for known Trans factors under conditions that promote protein-DNA binding. Protein binding to each DNA molecule is monitored by changes in fluorescence anisotropy values for labeled DNA fragments over time. Those fragments that shown an increase in fluorescence anisotropy values over time are scored as positives for protein binding. The greater and more rapid the increase, the lower the Kd for the protein/DNA complex. Thus, since the Kd is inversely proportional to the protein binding activity, which itself is dependent upon both protein concentration and affinity of the protein for its DNA binding site, the level of binding activity for each individual complex in each protein population can be quantitated.

In the above example, if nuclear extract from resting Jurkat cells is added to plate A and nuclear extract from TPA/ionomycin-activated Jurkat cells is added to plate B, one would expect to see a significant increase in the fluorescence anisotropy from the Ap-1 and NF-kB CIS element-containing DNA molecules in plate B compared to plate A due to known induction of both Ap-1 and NF-kB binding activities upon Jurkat cell activation with TPA/ionomycin. In contrast, one would expect to see a rapid and significant increase in the fluorescence anisotropy of the labeled Oct-1 and Sp-1 fragments in both plates A and B equally, due to the moderately high constitutive levels of Oct-1 and Sp-1 binding activities in both resting and activated Jurkat cells. Of importance, whether the binding activities are differentially active or constitutive between two cell populations, the global profiling methods described in this invention allow quantification of the levels of binding activities.

By comparison of assays performed as described in this example, a global profile of gene expression activation is discerned, allowing the mapping of gene regulation throughout a population of cells at any given metabolic or physiologic state.

A further example of determining global differences in gene regulatory element activity is illustrated by the representative data in Table 1. In this example, global profile's in resting Jurkat cells versus TPA-activated Jurkat cells were obtained by sequence analysis of the DNA fragments isolated as a result of being bound by sequence-specific binding proteins. DNA sequences were analyzed for known binding CIS elements and their occurrences quantified (expressed as a percentage of the total fragments analyzed). The degree to which any given CIS element is observed is a measure of relative binding activity within the original cell population, and can be compared to other CIS elements within the same cell population as well as to other cell populations. It can be seen from the subset of profile data (i.e., a partial profile) shown that certain binding sites are constitutive (similar levels between the two cell populations), while others are significantly differential in their level of activity. For example, the binding sites for transcription factor complexes CREB and NFAT each show a significant increase in binding activity, both of which have been associated with T-cell activation. Activation of T cells is a hallmark of certain immune disorders, including inflammation, allergy autoimmune diseases, tissue rejection and HIV-related diseases. The reduced binding activity of ARARNT, on the other hand, has not been reported previously.

TABLE 1

| | Resting Jurkat | Activated Jurkat |
|---|---|---|
| | % of total frags | % of total frags |
| ARARNT | 4.1 | 1.5 |
| AP1 C | 0.7 | 1.5 |
| AP-2 Q6 | 0.7 | 1.5 |
| ATF | 0.0 | 0.8 |
| CREB | 0.7 | 3.1 |
| CAAT | 6.8 | 5.4 |
| CETS1P54 | 2.7 | 2.3 |
| CMYB | 3.4 | 3.1 |
| CDPCR3 | 2.0 | 0.8 |
| E47 | 0.0 | 2.3 |
| MYCMAX | 1.4 | 3.8 |
| MZF1 | 12.8 | 10.0 |
| NFAT | 2.0 | 6.2 |
| NFY | 7.4 | 8.5 |
| NGF1C | 2.0 | 3.1 |
| Sp1 | 4.7 | 6.9 |
| GC | 3.4 | 6.9 |
| USF | 6.8 | 5.4 |
| WT1 | 1.4 | 7.7 |
| XBP1 | 1.4 | 3.1 |

Another CIS element/transcription factor complex found to exhibit differential binding activity between resting and activated Jurkat cells was NF-kB (binding activity via global profiling was carried out, but not shown here). Higher levels of NF-kB have also been found in activated T cells relative to resting cells and associated with T cell diseases such as those listed above. Furthermore, NF-kB has been shown to regulate genes important in T cell activation, such as numerous genes coding for cytokines. Confirmation of the increased binding activity in activated T cells was demonstrated by electrophoretic mobility shift assay. (EMSA). Nuclear extracts obtained from both resting Jurkat cells and TPA-activated Jurkat cells were added to separate binding reactions. Each reaction also contained a ³²P-labeled oligonucleotide comprising the binding site for NF-kB. Some reactions also contained competitor oligonucleotides. As shown in FIG. 2 (lanes 1-3), no labeled oligonucleotide shifted in the lanes from binding reactions containing resting Jurkat cell extract. As expected, no difference was seen between the lanes containing no competitor oligo (lane 1), a competitor oligonucleotide specific for the NF-KB binding site (lane 2), and a competitor oligonucleotide mismatched to the NF-kB binding site (lane 3). In contrast, a significant amount of gel-shifted material (DNA/protein complexes) was observed in lanes 4 and 6, which came from binding reactions containing nuclear extract from activated Jurkat cells. Lane 4 contained no competitor oligonucleotide and lane 6 contained mismatched competitor oligonucleotide. Lane 5, which contained matched competitor oligonucleotide, also showed no gel-shifted material, demonstrating specificity of the shifted DNA/protein complexes.

In another example, rat pheochromocytoma cells (cell line PC12) were grown in the presence of high serum, and then transferred to a serum-free medium containing 200 ng/ml Nerve Growth Factor-beta (NGF-beta). After 5 hr exposure, cells were harvested and nuclear protein extracts prepared. Global regulatory element activity profiling was carried out using nuclear extracts from cells either treated with NGF-beta or not treated. Known transcription factor binding sites present in the DNA sequences were counted as described for Jurkat cells, and the data generated are presented in FIG. 3. Bars indicate the percentage of DNA fragments containing selected CIS sites that were isolated in binding reactions containing nuclear extracts from either untreated (white bars) or NGFbeta-treated (black bars) PC12 cells. It can be seen that NGF treatment leads to an increase in binding activity for AP1, ATF, TCF11 (among others), while other activities, for example, E2F and FRX1, are reduced after NGF treatment. This analysis suggests that genes regulated by AP1, ATF or TCF11 may be activated upon NGF treatment, while genes regulated by E2F and RFX1 may be repressed upon NGF treatment. These results in PC12 cells involving the activity of specific CIS/Trans complexes and their ability to regulate gene expression are related to diseases involving neuronal cell death and regeneration. For example, in the PC12 model, AP-1 expression is associated with neurite outgrowth and protection from apoptosis (Dragunow et al, 2000. Brain Res Mol Brain Res 83:20-33). Relevant human diseases may involve either acute injury or chronic neuronal changes. Thus, the profiling of the present invention provides a real world application for identifying the regulatory effects of disease related molecules.

Confirmation of the increase in the transcription factor AP-1 binding activity was confirmed by electrophoretic mobility shift assay (EMSA). Nuclear extracts obtained from PC cells either treated with NGFbeta or untreated were combined in separate binding reactions. Each reaction also contained a ³²P-labeled oligonucleotide comprising a binding site for a specific transcription factor. As shown in FIG. 4 (lanes 3 and 4), a significant increase in the gel-shifted material (DNA/protein complexes) was observed in the NGF-treated cells when the oligonucleotide was specific for the AP-1 binding site. In contrast, when the oligonucleotide was specific for the OCT1 binding site (lanes 7 and 8), no increase in gel-shifted material was observed. These results demonstrate again that AP-1 is increased in binding activity to its CIS sequence in PC12 cells treated with NGFbeta. They also demonstrate that OCT1 CIS/Trans complexes are present in both cell populations but not differential between the NGF-treated and untreated cells.

These results illustrate the feasibility as well as the usefulness of determining global gene regulatory element activity profiles involving quantitative levels of CIS element/Trans factor binding activities within cell populations. These profiles can then be compared between different cell populations to discern differences in gene expression important in overall genetic and phenotypic changes. As is clear to those skilled in the art, identification of such changes, as determined by the global profiling of the present invention, is useful in many applications in medicine, such as determining the effects of compounds on gene regulation, and recognition of disease states in cells. FIG. 5 provides an example schematic of the profile determining process of the invention.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted for the global profiling of gene regulatory element activity. The specific methods and compositions described herein as presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention. For example, those skilled in the art will recognize that the invention may suitably be practiced using a variety of laboratory protocols to obtain useful data respecting globally profiled regulatory elements.

The invention illustratively described herein suitably may be practiced in the absence of any element or elements, limitation or limitations which are not specifically disclosed herein as essential. Thus, for example, in each instance herein, in embodiments of the present invention, any of the terms “comprising,” “consisting essentially of” and “consisting of” may be replaced with either of the other two terms. The terms and expressions that have been employed are used as terms of description and not of limitation, and there is not intention, in the use of such terms and expressions, of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. For example, if there are alternatives A, B, and C, all of the following possibilities are included: A separately, B separately, C separately, A and B, A and C, B and C, and A and B and C. Thus, the embodiments expressly include any subset or subgroup of those alternatives. While each such subset or subgroup could be listed separately, for the sake of brevity, such a listing is replaced by the present description.

While certain embodiments and examples have been used to describe the present invention, many variations are possible and are within the spirit and scope of the invention. Such variations will be apparent to those skilled in the art upon inspection of the specification and claims herein.

Other embodiments are within the following claims.

### REFERENCES

Struhl, K. (1995) Annu. Rev. Genet. 29:651-674.

Ptashne, M. and Gann, A. (1997) Nature 386:569-577.

Orphanides et al. (1996) Genes Dev. 10:2657-2683.

Roeder R. G. (1996) Trends Biochem. Sci. 21:327-335.

Myer, V. and Young, R. A. (1998) J. Biol. Chem. 273-27757-27760.

Burely, S. K. and Roeder, R. G. (1996) Annu. Rev. Biochem. 65:769-799.

Roth, S. Y. and Allis, C. D. (1996) Cell 87:5-8.

Steger, D. S. and Worlanan, J. L. (1996) Bioessays 18:875-884;

Fields and stemglanz (1994) trends genet. 10:286-292.

Harris, M. (1998) methods mol. Biol. 88:87-99.

Velculescu et al. (1995) science 270:484-487.

Schena et al. (1995) science 270:467-470.

Chee et al. (1996) science 274:610-614.

Lakowicz J. R. Pricipals of Fluorescence spectroscopy; Plenum Press: New York, 1983.

Dandiker and deSaussure, (1970) Immunochemistry 7:799-828.

Chee et. al. (1996) Science 274:610-614.

Lockhart et al. (1996) Nat. Biotechnol. 14:1675-1680.

DeRisi, J. L. et al. (1997) Science 278:680-686.

Holstege F. C. et al. (1998) Cell 95:717-728.

Wodicka, L. et al. (1997) Nature Biotechnol. 15:1359-1367.

DeRisi J. L. et al. (1996) Nature Genet. 14:457-460.

Alizadeh, A. A. et al. (2000) Nature 403:503-511.

Lossos, I. S. et al. (2000) PNAS 97:10209-10213.

Schena, M. (1996) PNAS 93:10614-10619.

Gray, N. S. et. A1. (1998) Science 281:533-538.

Checovich, W. J. et al. (1995) Nature 375:254-256.

What is claimed is:

1. A method for global profiling regulatory element activity in a host cell comprising:
a) providing a source of CIS element-containing nucleic acid sequences;
b) providing from said host cell a source of cellular proteins from a first and at least a second physiologic and/or metabolic state;
c) contacting said CIS element containing nucleic acid of (a) with said cellular proteins of (b), wherein said contacting provides for formation of protein/nucleic acid complexes between said CIS element containing nucleic acid and said cellular proteins for each of said first and at least second physiologic or metabolic state;
d) detecting said complexes;
e) identifying either or both an amino acid sequence of said cellular proteins and a nucleic acid sequence of said CIS element-containing nucleic acid of said complexes of (d); and
f) comparing said complexes of (d) and nucleic acid sequences and/or amino acid sequences of (e) of said first physiologic or metabolic state with said at least second physiologic or metabolic state, such that a global profile of said regulatory element activity is obtained.

2. A method according to claim 1 wherein said source of CIS element containing nucleic acid is selected from a cell, a preparation of genomic nucleic acid, and a library of synthetically prepared nucleic acid.

3. A method according to claim 1 wherein said source of cellular proteins is selected from a total cellular extract and a nuclear cellular extract of said host cell.

4. A method according to claim 1 wherein said detecting comprises use of fluorescent polarization.

5. A method according to claim 1 wherein said global profiling is carried out for determining a difference between gene expression regulation at one cellular metabolic state and a gene expression regulation at a second metabolic state.

6. A method according to claim 1 wherein said complexes are separated from nucleic acids not forming said complexes before said detecting of complexes is carried out.

7. A method according to claim 6 wherein said separation is carried out by a method selected from the group consisting of EMSA electrophoresis, capillary electrophoresis (CE), filtration, affinity purification, enzymatic digestion, and centrifugation.

8. A method according to claim 7 wherein said filtration uses size exclusion filters.

9. A method according to claim 7 wherein said digestion is digestion of nucleic acid not forming said complexes.

10. A method according to claim 1 wherein said CIS containing nucleic acid is in contact with a surface comprising a microarray.

11. A method according to claim 1 wherein said detection comprises direct detection of said complexes wherein said direct detection makes use of a label selected from the group consisting of a fluorescent label and a chemiluminescent label.

12. A method according to claim 1 wherein said detection comprises direct detection wherein said complexes are detected in the presence of nucleic acid and protein not forming said complexes.

13. A method according to claim 4 wherein said detection comprises direct detection wherein said complexes are detected in the presence of nucleic acid and protein not forming said complexes.

14. A method according to claim 1 wherein said detection comprises a separation of said complexes from nucleic acids and proteins not forming said complexes.

15. A method according to claim 13 wherein said detection further comprises a quantification of complexes.

16. A method according to claim 1 wherein said detection further provides for determination of CIS elements.
